(19) United States
(12) Patent Application Publication
(10) Pub. No.: US 2024/0374290 A1
(43) Pub. Date: Nov. 14, 2024

(54) Dead Foot-Skin Removal Device

(71) Applicant: Timothy Weaver, Rogers, AR (US)

(72) Inventor: Timothy Weaver, Rogers, AR (US)

(21) Appl. No.: 18/416,351

(22) Filed: Jan. 18, 2024

Related U.S. Application Data
(60) Provisional application No. 63/500,755, filed on May 8, 2023.

(51) Int. Cl. A61B 17/54; A61H 23/00

(52) U.S. Cl. CPC A61B 17/54; A61H 23/00; A61H 2201/0207; A61H 2201/1642; A61H 2205/12

(57) ABSTRACT

A dead skin removal and callus removal device is disclosed that comprises a base component with an exterior shell molded with plastic or silicone. The interior of the device is comprised of a scrubbing tool that may be used in the shower or bathtub. Users can easily secure the dead skin removal device to the base of a shower or bathtub using the attached suction cups on the base of the device. The device can be constructed of an antimicrobial plastic or silicone that inhibits the growth of unwanted bacteria and other fungi that may cause additional health concerns. In addition, the base component comprises a small pull tab to conveniently remove the device from a secured position for ease of cleaning and reuse.

### CROSS-REFERENCE TO RELATED APPLICATION

The present application claims priority to, and the benefit of, U.S. Provisional Application No. 63/500,755, which was filed on May 8, 2023, and is incorporated herein by reference in its entirety.

### FIELD OF THE INVENTION

The present invention relates generally to the field of dead skin removal. More specifically, the present invention relates to an improved soft skin device to facilitate the removal of dead skin from the bottom or heels of feet. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to the improvements in dead skin and callus removal from feet and heels. Generally, individuals with limited mobility and dexterity may lack the ability to reach the bottoms of their feet or heels to remove calluses. To remove calluses, individuals typically use a file or emery board to remove the hardened skin. However, this method may be messy and uncomfortable. In addition, many individuals with limited mobility and flexibility do not have the ability to remove the dead skin located on the bottom of their feet or heels independently.

With the increase in the number of individuals with diabetes or other health conditions that cause poor blood flow to the feet, which puts said individuals at greater risk of complications from calluses, the development of new callus-removing products is needed. While wearing shoes that fit properly, socks, and avoiding high heels are preventative measures to reduce the development of calluses, the development of dead skin and calluses is unavoidable. Generally, especially with regard to people with limited mobility and dexterity, diabetes, or other health conditions, users need a dead skin removal device that allows them to reach the bottom of their feet independently.

Therefore, there exists a long-felt need in the art for a device that allows a user with limited mobility, flexibility, and dexterity to remove dead skin or calluses from the bottom of their feet. Moreover, there exists a long-felt need in the art for a dead foot-skin removal device that allows users to remove dead skin and calluses from the bottom of their feet without having to reach or bend to a position in which their body will not allow. Further, there exists a long-felt need in the art for a dead foot-skin removal device that is compact and can be secured to a variety of surfaces for use.

The subject matter disclosed and claimed herein, in one embodiment, thereof, comprises a dead foot-skin removal device. The device is designed to remove dead skin and calluses from the bottom of feet and heels. The device is comprised of a base component having an exterior shell molded. The interior of the device is comprised of a scrubbing tool that may be used in a shower or a bathtub. During use, users can easily secure the device to the base of a shower or bathtub using attached suction cups on the base. The device can also be constructed of an antimicrobial plastic or silicone that inhibits the growth of unwanted bacteria and other fungi that may cause additional health concerns. In addition, the base component comprises a small pull tab to conveniently remove the device from a secured position for ease of cleaning and reuse.

In this manner, the skin removal device of the present invention accomplishes all of the foregoing objectives and provides users with a compact and lightweight callus removal system. In addition to users with limited flexibility and dexterity, the device can be used by anyone with dead skin or calluses on the bottom or heels of their feet. Furthermore, the antimicrobial plastic or silicone construction of the device makes skin removal desirable to users of all types.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a dead foot-skin removal device. The device provides users with limited mobility and flexibility the ability to remove dead skin from the bottom of their feet and heels safely. The device is preferably made from a plastic or silicone antimicrobial material shell with at least one scrubbing tool positioned on the interior of the device. Users can secure the device to the floor of a shower or bathtub. The device allows users with limited flexibility or dexterity to remove dead skin or calluses from the bottom of their feet or heels without the use of messy and cumbersome tools.

Specifically, the device comprises an outer shell base component, two side components, a back side component, and a front opening. The base component shell may be manufactured of an antimicrobial molded plastic or silicone that inhibits the growth of unwanted bacteria and other fungi that may cause additional health concerns.

During use, a user can secure the device to the base of a bathtub, shower, or other surface using at least one fastener (preferably a suction cup) located anywhere on the base of the outer shell. To aid in removing the device from a surface after it has been attached by the fasteners, the device may be comprised of at least one pull tab. After placing a foot into the device such that it contacts at least one coarse scrubbing tool and/or at least one fine scrubbing tool, a user can move their foot in a back and forward motion to remove dead skin and calluses from the bottom of the foot while pressing against the coarse scrubbing tool or the fine scrubbing tool.

The scrubbing tools are preferably in the form of a textured liner that can be placed inside the device. While the foot is pressed against the back side of the device, the foot can be moved in an up-and-down motion to remove dead skin and calluses from the heel of the foot. Then, the user will remove the coarse scrubbing tool and replace it with the fine scrubbing tool (of the same liner-like construction) to polish the bottom of the foot or heel to their desired level.

The present invention is also comprised of a method of using the device. First, a device is provided comprised of an interior space that receives at least one insert and an outer base shell comprised of at least one fastener. Then, a user can place and secure the insert within the interior space of the device via at least one fastener of the interior space. Then, a user can secure the device to a surface using the fastener. Next, a user can insert their foot into the interior space and move their foot forward and backward along the insert to remove dead skin from their foot. Optionally, a user can add water to the insert while moving their foot to improve performance of the insert.

Numerous benefits and advantages of this invention will become apparent to those skilled in the art to which it pertains, upon reading and understanding the following detailed specifications.

To the accomplishment of the foregoing and related ends, certain illustrative aspects of the disclosed innovation are described herein in connection with the following description and the annexed drawings. These aspects are indicative, however, of but a few of the various ways in which the principles are disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which;

FIG. 1 illustrates multiple perspective views of one potential embodiment of a dead foot-skin removal device of the present invention in accordance with the disclosed architecture;

FIG. 2 illustrates a perspective view of one potential embodiment of a dead skin foot removal device of the present invention with a user's foot placed within the device in accordance with the disclosed architecture;

FIG. 3 illustrates a side view and rear perspective view of one potential embodiment of a dead foot-skin removal device of the present invention in accordance with the disclosed architecture;

FIG. 4 illustrates a side cross-sectional view of one potential embodiment of a dead foot-skin removal device of the present invention in accordance with the disclosed architecture;

FIG. 5 illustrates a perspective view of one potential embodiment of a dead foot-skin removal device of the present invention while attached to a surface in accordance with the disclosed architecture; and

FIG. 6 illustrates a flowchart showing the method of operating and storing the soft skin device in accordance with the disclosed architecture.

### DETAILED DESCRIPTION OF THE PRESENT INVENTION

The innovation is now described with reference to the drawings, wherein like reference numerals are used to refer to like elements throughout. In the following descriptions, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein form different embodiments may be combined.

As noted above, there exists a long-felt need in the art for a device that allows a user with limited mobility, flexibility, and dexterity to remove dead skin or calluses from the bottom of their feet. Moreover, there exists a long-felt need in the art for a dead foot-skin removal device that allows users to remove dead skin and calluses from the bottom of their feet without having to reach or bend to a position in which their body will not allow. Further, there exists a long-felt need in the art for a dead foot-skin removal device that is compact and can be secured to a variety of surfaces for use.

The present invention, in one exemplary embodiment, is comprised of a dead foot-skin removal device. The device provides users with limited mobility and flexibility the ability to remove dead skin from the bottom of their feet and heels safely. The device is preferably made from a plastic or silicone antimicrobial material shell with at least one scrubbing tool positioned on the interior of the device. During use, users can secure the device to the floor of a shower or bathtub. The device allows users with limited flexibility or dexterity to remove dead skin or calluses from the bottom of their feet or heels without the use of messy and cumbersome tools.

The device has an outer shell base component, two side components, a back side component, and a front opening. The base component shell may be manufactured of an antimicrobial molded plastic or silicone. Said material inhibits the growth of unwanted bacteria and other fungi that may cause additional health concerns.

During use, a user can secure the device to the base of a bathtub, shower, or other surface using at least one fastener (preferably a suction cup) located anywhere on the base of the outer shell. To aid in removing the device from a surface after it has been attached by the fasteners, the device may be comprised of at least one pull tab. After placing a foot into the device such that it contacts at least one coarse scrubbing tool and/or at least one fine scrubbing tool, a user can move their foot in a back and forward motion to remove dead skin and calluses from the bottom of the foot while pressing against the coarse scrubbing tool or the fine scrubbing tool.

The scrubbing tools are preferably in the form of a textured liner that can be placed inside the device. While the foot is pressed against the back side of the device, the foot can be moved in an up-and-down motion to remove dead skin and calluses from the heel of the foot. Then, the user will remove the coarse scrubbing tool and replace it with the fine scrubbing tool (of the same liner-like construction) to polish the bottom of the foot or heel to their desired level.

The present invention is also comprised of a method of using the device. First, a device is provided comprised of an interior space that receives at least one insert and an outer base shell comprised of at least one fastener. Then, a user can place and secure the insert within the interior space of the device via at least one fastener of the interior space. Then, a user can secure the device to a surface using the fastener. Next, a user can insert their foot into the interior space and move their foot forward and backward along the insert to remove dead skin from their foot. Optionally, a user can add water to the insert while moving their foot to improve performance of the insert.

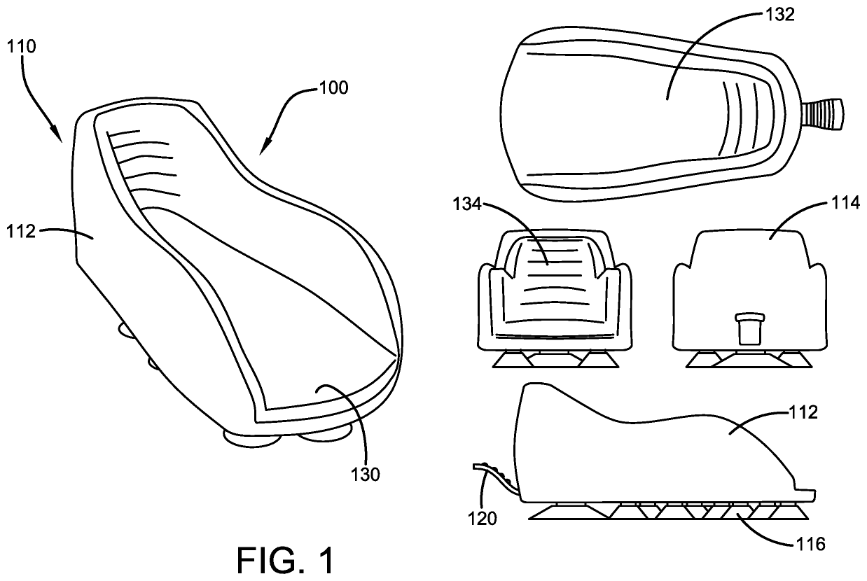

Referring initially to the drawings, FIG. 1 illustrates a perspective view of one embodiment of the soft skin device 100 of the present invention. The device 100 provides users with limited flexibility and dexterity the ability to care for their feet independently. Specifically, the device 100 comprises an outer shell base component 110, two side components 112, a back side component 114, and a front opening 115. During use, a user can secure the device 100 to the base of a bathtub, shower, or other surface using at least one suction cup 116 located anywhere on the base of the outer shell 110. The device 100 easily secures to a shower base, bathtub base, or any other suitable flat surface for versatility.

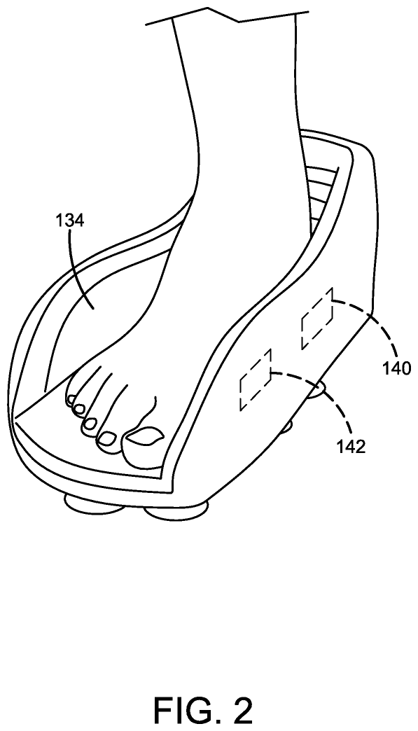

After placing a foot into the device 100 such that it contacts at least one coarse scrubbing tool 130 and/or at least one fine scrubbing tool 132 (made from any skin-removing material or structure known in the art), a user can move their foot in a back and forward motion to remove dead skin and calluses from the bottom of the foot while pressing against the coarse scrubbing tool 130 or the fine scrubbing tool 132, as seen in FIG. 2. Generally, a user can first use the coarse scrubbing tool 130 to remove the dead skin to their desired level. The tool 130 is preferably in the form of a textured liner that can be placed inside the device 100. While the foot is pressed against the back side 114 of the device 100, the foot can be moved in an up-and-down motion to remove dead skin and calluses from the heel of the foot. Then, the user will remove the coarse scrubbing tool 130 and replace it with the fine scrubbing tool 132 (of the same liner-like construction) to polish the bottom of the foot or heel to their desired level. Typically, users will repeat this process over an extended time regularly, depending on the level of dry skin and calluses. The tools 130, 132 may attach to the interior space 102 of the device 100 via at least one fastener 104 such as, but not limited to, snap button, hook and loop, magnet, adhesive, etc.

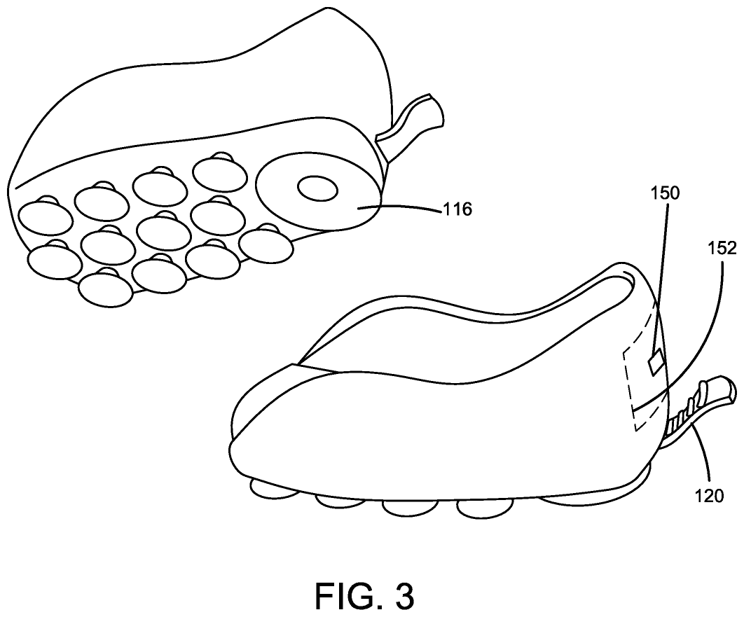

The device 100 may also be comprised of at least one fastener 116 such as, but not limited to, a suction cup, a magnet, hook and loop, etc., as seen in FIG. 3. The fastener 116 allows the device 100 to attach to any surface such as, but not limited to, a bathtub, wall, floor, etc., to keep the device 100 in place during use. To aid in removing the device 100 from a surface after it has been attached by the fasteners 116, the device 100 may be comprised of at least one pull tab 120. In an embodiment where a user has limited flexibility and dexterity and lacks the ability to reach the tab 120 from a seated or standing position, at least one detachable arm extension 122 removably attaches to the tab 120 via at least one fastener 124 such as, but not limited to, a clip or a clamp. A user can then pull the extension 122 upwards after attachment to the pull tab 120 to remove the device 100 from a surface.

Alternatively, the interior space 102 may receive a massaging insert 134 that attaches to the interior space 102 via the fastener 104. The insert 134 may have any massaging structure known in the art. In one embodiment, any combination of elements 130, 132, 134 may be on a singular or differing liner. At least one heating element 140 and/or vibrating element 142 may also be present in the interior surface 102. The heating element 140 and vibrating element 142 provide additional health benefits, such as, increasing blood flow and softening the skin on the foot. The elements 140, 142 are powered by at least one battery 152. The battery 152 may be rechargeable or disposable. In one embodiment, the battery 152 can be recharged via at least one charging port 150 of any type known in the art and/or power cord.

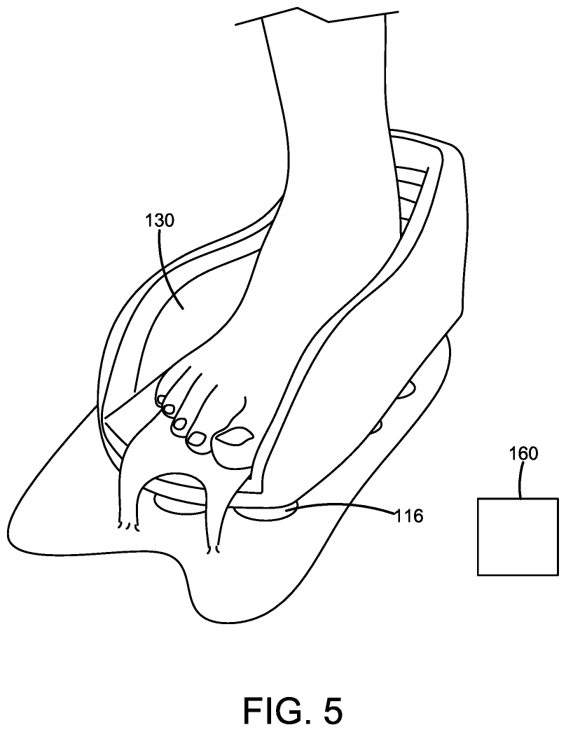

In the present embodiment, the soft skin device 100 is an improved dead skin or callus removal device that illustrates a user standing in the device 100 while in the shower, as seen in FIG. 5. Typically, wet or soaked skin improves the ability to remove dead skin or calluses on the foot. Generally, users will utilize the device 100 in the shower or bathtub when the skin is wet to maximize the results of the soft skin device 100.

In addition to the added features, in one embodiment, the device 100 has a carrying case 160 with handles for carrying and storage. Typically, when the soft skin device is not in use, the user can use the handles to hang the device 100 on the shower or bathtub wall for convenience and ease of access.

Furthermore, the base component shell 110 may be manufactured of an antimicrobial molded plastic or silicone that inhibits the growth of unwanted bacteria and other fungi that may cause additional health concerns. The device 100, constructed of a strong, durable plastic or silicone material, makes cleaning and sanitizing simple and safe.

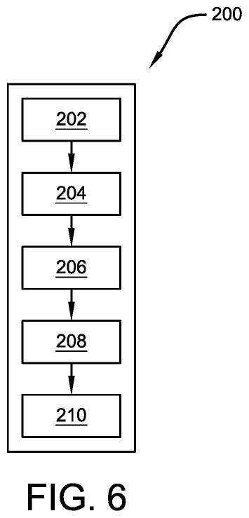

FIG. 6 illustrates a flowchart of the method 200 of using the device 100. First, a device is provided comprised of an interior space 102 that receives at least one insert 132, 134 and an outer base shell 110 comprised of at least one fastener 116 [Step 202]. Then, a user can place and secure the insert 132, 134 within the interior space 102 of the device 100 via at least one fastener 104 of the interior space 102 [Step 204]. Then, a user can secure the device 100 to a surface using the fastener 116 [Step 206]. Next, a user can insert their foot into the interior space 102 and move their foot forward and backward along the insert 132, 134 to remove dead skin from their foot [Step 208]. Optionally, a user can add way to the insert 132, 134 while moving their foot to improve the performance of the insert 132, 134 [Step 210].

Certain terms are used throughout the following description and claims to refer to particular features or components. As one skilled in the art will appreciate, different users may refer to the same feature or component by different names. This document does not intend to distinguish between components or features that differ in name but not structure or function. As used herein “soft skin device”, “dead skin removal device”, “callus removal device” and “device” are interchangeable and refer to the soft skin device 100 of the present invention.

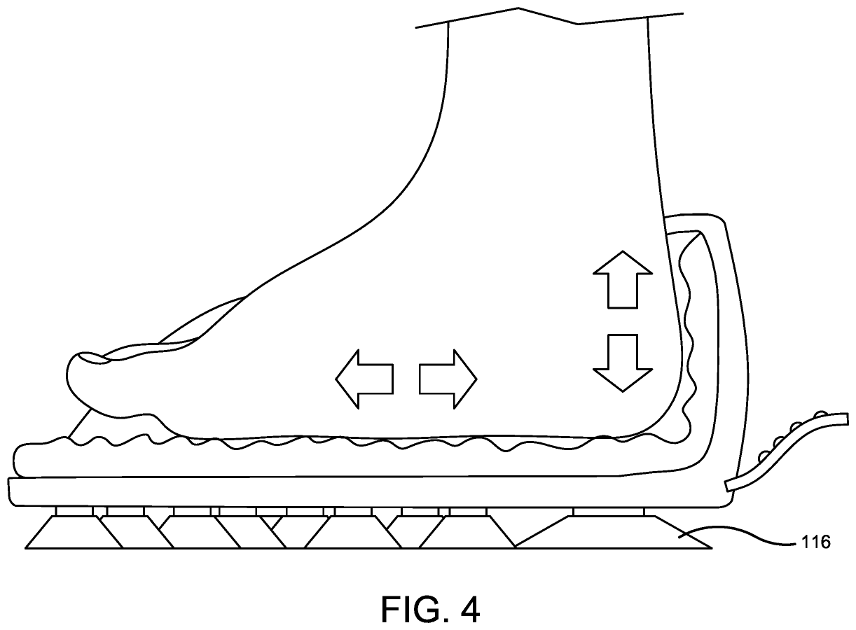

Notwithstanding the foregoing, the soft skin device 100 of the present invention can be of any suitable size and configuration as is known in the art without affecting the overall concept of the invention, provided that it accomplishes the above-stated objective. One of ordinary skill in the art will appreciate that the soft skin 100 as shown in FIGS. 1-6 is for illustrative purposes only, and that many other sizes of the soft skin device 100 are well within the scope of the present disclosure. Although the dimensions of the soft skin device 100 are important design parameters for user convenience, the dead skin removal device 100 may be of any size that accommodates the user and ensures optimal performance during use.

Various modifications and additions can be made to the exemplary embodiments discussed without departing from the scope of the present invention. While embodiments described above refer to particular features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or claims, such term is intended to be inclusive in a manner similar to the term “comprising” as “comprising” is interpreted when employed as a transitional word in a claim.

What is claimed is:

1. A dead foot-skin removal device comprising:
an outer base shell;
a coarse scrubbing tool;
a fine scrubbing tool; and
a massaging insert.

2. The dead foot-skin removal device of claim 1, wherein the outer base shell is comprised of a fastener.

3. The dead foot-skin removal device of claim 2, wherein the fastener is comprised of a suction cup.

4. The dead foot-skin removal device of claim 1, wherein the coarse scrubbing tool is comprised of a liner that can be placed into the outer base shell.

5. The dead foot-skin removal device of claim 4, wherein the coarse scrubbing tool attaches to the outer base shell via a first fastener.

6. The dead foot-skin removal device of claim 1, wherein the fine scrubbing tool is comprised of a liner that can be placed into the outer base shell.

7. The dead foot-skin removal device of claim 6, wherein the coarse scrubbing tool attaches to the outer base shell via a second fastener.

8. The dead foot-skin removal device of claim 1 further comprised of a pull tab.

9. The dead foot-skin removal device of claim 1, wherein the base shell is comprised of an anti-microbial material.

10. A dead foot-skin removal device comprising:
an outer base shell;
a coarse scrubbing tool;
a fine scrubbing tool;
a massaging insert;
a heating element;
a vibrating element; and
a battery.

11. The dead foot-skin removal device of claim 10, wherein the outer base shell is comprised of a fastener.

12. The dead foot-skin removal device of claim 11, wherein the fastener is comprised of a suction cup.

13. The dead foot-skin removal device of claim 10, wherein the coarse scrubbing tool is comprised of a liner that can be placed into the outer base shell.

14. The dead foot-skin removal device of claim 13, wherein the coarse scrubbing tool attaches to the outer base shell via a first fastener.

15. The dead foot-skin removal device of claim 10, wherein the fine scrubbing tool is comprised of a liner that can be placed into the outer base shell.

16. The dead foot-skin removal device of claim 15, wherein the coarse scrubbing tool attaches to the outer base shell via a second fastener.

17. The dead foot-skin removal device of claim 10, wherein the base shell is comprised of an anti-microbial material.

18. The dead foot-skin removal device of claim 10, wherein the battery is comprised of a rechargeable battery.

19. The dead foot-skin removal device of claim 18, wherein the battery is comprised of a charging port.

20. A method of using a dead foot-skin removal device, the method comprising the following steps:
providing a dead foot-skin removal device comprised of an interior space that receives an insert and an outer base shell comprised of a fastener;
placing the insert within the interior space;
securing the dead foot-skin removal device to a surface via the fastener; and
inserting a foot into the interior space and moving the foot forwards and backwards along the insert to remove a dead skin from the foot.
